(19) United States
(12) Patent Application Publication
(10) Pub. No.: US 2017/0246217 A1
(43) Pub. Date: Aug. 31, 2017

(54) USE OF CORD BLOOD PLASMA TO TREAT NK CELL-MEDIATED DISEASES AND IFN-&#947; MEDIATED DISEASES

(71) Applicants: Funndación Pública Andaluza Progreso y Salud, Sevilla (ES); Anthony Nolan The Royal Free Hospital, London, Hampstead (GB); Instituto de Salud Carlos III, Madrid (ES)

(72) Inventors: Bernat SORIA ESCOMS, Sevilla (ES); Abdelkrim HMADCHA, Sevilla (ES); José Alejandro MADRIGAL, London, Hampstead (GB); Aurore SAUDEMONT, London, Hampstead (GB); Steven COX, London, Hampstead (GB); Raquel LAZA, London, Hampstead (GB)

(21) Appl. No.: 15/511,605

(22) PCT Filed: Sep. 16, 2015
(86) PCT No.: PCT/EP2015/071252

§ 371 Date: Mar. 15, 2017

(30) Foreign Application Priority Data
Sep. 16, 2014 (EP) 14382348.2

(51) Int. Cl. A61K 35/51

(52) U.S. Cl. CPC A61K 35/51

(57) ABSTRACT

The present invention shows that CB plasma contains soluble NKG2D ligands and that the incubation of PBMCs with CB plasma results in decreased cytotoxicity, decreased proliferation and inhibition of IFNy production by NKG2D bearing cells, in particular, NK cells. Interaction of NKG2D with soluble NKG2D ligand leads to blocking of the NKG2D receptor, and renders NKG2D bearing cells refractory to activation and inhibits cell functions. Notably, this is a mechanism naturally employed by tumor cells or viruses such as CMV to escape the immune system.

### FIELD OF THE INVENTION

The present invention relates to the field of immunology and medicine, particularly to the cosmetics/dermatological and medical treatments, and more specifically to the use of cord blood plasma in the prevention, treatment or amelioration of NK cell-mediated diseases and/or IFN-γ mediated diseases. In particular, the present invention provides a cord blood plasma composition for use in preventing, treating or ameliorating one or more symptoms associated with disorders in which modulation of a subject's immune system is beneficial, including, but not limited to, autoimmune diseases, inflammatory disorders, and immunologically mediated diseases including rejection of transplanted organs and tissues.

### BACKGROUND OF THE INVENTION

Natural Killer (NK) cells are effector lymphocytes of the innate immune system that do not need MHC-I recognition. NK cells can detect a lack of class I molecules on the infected cell surface, referred to as ‘missing self’, due to down-regulation by the pathogen. This situation alerts the NK cell of the possible presence of a virus and the infected cell is lysed by perforin, which is released by the NK cell, together with granzymes that enter the cell leading to apoptosis. NK cells are potent producers of IFN-γ when activated that furthers an inflammatory response by recruiting or activating other effector cells. NK cells are innate immune cells originally identified by their ability to lyse certain tumour cell lines without prior stimulation in vitro or in vivo. These cells provide a first line of defence against not only transformed cells but also virally infected cells. Viruses have evolved to evade the immune response in many different ways, including down-regulation of ‘self’ major histocompatibility complex (MHC) class I molecules to avoid detection by CD8+ cytotoxic T cells. NK cells are also equipped with activating receptors that can interact with conserved ‘non-self’ molecules on microbes, leading to direct lysis. Another mechanism referred to as ‘altered self’ involves interaction of NKG2D activating receptor with ligands that are up-regulated on infected cells, leading to apoptosis. Recent findings support the hypothesis that NK cells are also regulators of dendritic cells, macrophages, T-cells and endothelial cells thus regulating (either limiting or increasing) immune responses. Pharmacological control of NK cells may be useful in controlling inflammatory and autoimmune disorders. In humans NK cells have been shown to home inflamed skin (Parolini S et al., 2007. Blood 109: 3625-3632) in various conditions such as vernal keratoconjunctivitis (Lambiase A et al., 2007. Mol Vis 13: 1562-1567), atopic dermatitis Buentke E et al., 2002. J Invest Dermatol 119: 850-857), psoriasis (Ottaviani C et al., 2006. Eur J Immunol 36: 118-128) or lichenplanus (Tagliabue et al., 1982. J Exp Med 155: 1785-1796). NK cells have been also detected in the gut (Geremia et al. 2014, Autoimmun Rev 13(1): 3-10). NK cells are effector lymphocytes, which are recruited upon inflammation by the effect of chemokine receptors CCR2, CCR5, CXCR3 and CX3CR17. Activating NK cell receptors detect the presence of ligands on cells in “distress” such as the stress-induced self-ligands recognized by NKG2D (human ULBI and MIC molecules (Lanier L L. 2005. Ann Rev Immunol. 23: 225-274).

T-cell antigen receptors recognize fragments of antigens bound to antigen-presenting molecules on the surface of antigen-presenting cells (dendritic cells). T-cell activation requires both the binding to antigen-presenting macromolecules and coactivation by costimulatory molecules. Dendritic cells are highly specialized in this task and skin contains a large number of dendritic cells, both in the epidermis (Langerhans cells) and in the dermis. Naïve T-cells, migrated from other areas of the body, activate in lymph nodes. After activation T-cells proliferate and express activation molecules and undergoe transition to memory T-cells. Memory T-cells in inflammatory skin diseases express CLA on their surface; in contrast T-cells in inflammatory diseases involving other tissues (gut, bone joints, etc) are predominantly CLA-negative. CLA-positive T-cells represent 10-15% of all circulating T-cells in peripheral blood. During cutaneous inflammation. E-selectin, the endogenous ligand for CLA, is highly expressed. This preferential expression helps to select for CLA T-cells under inflammatory conditions. After epithelial homing cytokines with broad effect on inflammation and immunity (interleulin-1, tumour necrosis factor alpha, interferon gamma, etc) are released by activated T-cells, and other immune system cells (NKT) maintaining a self-activating state of inflammation in the skin.

In summary, NK cells provide a first line of defence against transformed cells and virally infected cells.
Viruses can evade the immune response in many different ways, including down-regulation of MHC class I molecules to avoid detection by CD8+ cytotoxic T cells. However, NK cells can detect a lack of class I molecules on the infected cell surface; NK cells release perforin and with granzymes induces apoptosis in the infected cells.
When activated, NK cells are potent producers of IFN-γ that continues an inflammatory response by recruiting or activating other effector cells.
NK cells are also equipped with activating receptors that can interact with conserved ‘non-self’ molecules on microbes, leading to direct lysis.
Another mechanism referred to as ‘altered self’ involves interaction of NKG2D activating receptor with ligands that are up-regulated on infected cells, leading to apoptosis.

Indeed, decreased NK cell activity is associated with cancer and infectious diseases (see Yamazaki et al., 2002. Oncology Reports 9:359-363; Rosenberg et al., 1991. Cancer Research 51:5074-5079 (suppl.); Britteenden et al., 1996. Cancer 77:1226-1243, 1996; U.S. Pat. Nos. 5,082,833 and 4,883,662). Conversely, NK cell activity mediates acute rejection of BMC allografts. Therefore, levels of NK cell activity appear to play an important role in immune-related disorders.

In contrast to their protective role, NK cells can also act as mediators of innate immunopathology. In patients with chronic hepatitis B virus infection, a subset of NK cells contributes to liver inflammation by inducing hepatocyte death through a TRAIL-dependent mechanism (Dunn, C. et al., 2007. J. Exp. Med. 204, 667-680). In hepatitis B virus transgenic mice, NK cells also promote liver injury through NKG2D (Chen, Y. et al., 2007. Hepatology 46, 706-715). Moreover, NK cells act detrimentally in experimental sepsis induced by Streptococcus pneumoniae or Escherichia coli by exacerbating inflammatory responses (Kerr, A. R. et al., 2005. Microbes Infect. 7, 845-852; Badgwell, B. et al., 2002. Surgery 132, 205-212). In a mouse model of diabetes induced by coxsackie virus B4, NK cells contribute to beta-cell islet destruction (Flodstrom, M. et al., 2002. Nat. Immunol. 3, 373-382 (2002)). Consistent with these data, a significant increase of various NK cell transcripts is detected in destructive forms of the BDC2.5 mouse diabetes model (Poirot, L et al., 2004. Proc. Natl. Acad. Sci. USA 101, 8102-8107). A potential contribution of NK cells has also been postulated in human inflammatory diseases such as arthritis (de Matos, C. T. et al., 2007. Immunology 122, 291-301) and sarcoidosis (Katchar, K. et al., 2005. Eur. Respir. J. 26, 77-85).

Little is known regarding the presence and function of NK cells in epithelia. It has been recently reported that NK cells can mediate hapten-specific recall responses, independent of B cells and T cells, in a model of contact hypersensitivity (O'Leary, J. G. et al., 2006. Nat. Immunol. 7, 507-516). The involvement of NK cells in memory-type immune responses is quite unexpected and needs to be dissected in depth. Whether the skin infiltration involves hapten-specific NK cell receptors or whether various haptens induce distinct types of inflammation that do or do not promote NK cell infiltration is also unknown. In humans, NK cells have been shown to home to inflamed skin in various conditions, such as vernal keratoconjunctivitis (Lambiase, A. et al., 2007. Mol. Vis. 13, 1562-1567), atopic dermatitis (Buentke, E. et al., 2002. J. Invest. Dermatol. 119, 850-857), psoriasis (Ottaviani, C. et al., 2006. Eur. J. Immunol. 36, 118-128) and lichen planus. NK cells have also been detected in the gut (Tagliabue, A. et al., J. Exp. Med. 155, 1785-1796), but the physiological significance of these observations remains to be precisely addressed.

Patients undergoing hematopoietic progenitor transplantation (HSCT) cells undergo a prolonged period of immune dysfunction that can persist for several years. Patients have a predictable pattern of impairment and recovery of the immune system and there is an impairment of both cellular and humoral immunity.

Alloimmunity is a complex process which involves both T cells and NK cells from the donorinteracting with specific cell receptors. These cells are normally inhibited by negative signals through KIR receptors, which interact with MHC Class I molecules of target cells. When they find cells that have lost class I molecules or are not recognized as their own (as in non-HLA-matched transplants), the negative signal is produced, and the release of substances such as perforin and granzyme. Alloreactive NK cells also secrete proinflammatory cytokines IFN-γ and TNF-α to increase expression of MHC molecules and costimulatory receptors on the surface of APCs (antigen-presenting cells). This promotes APC maturation, which leads to amplification of T-cell alloreactivity by means of direct and also indirect pathway of alloantigen recognition.

Skin, as the primary interface between the body and the environment, is the subject for numerous injuries. Injuries translate into cutaneous inflammation, which includes innate immunity (Natural Killer, NK cells) and the recruitment of memory T-lymphocytes. Certain memory T cells appear to remember the anatomical site where they first encountered the antigen (Robert &Kupper T S. 1999. New England Journal of Medicine 341: 1817-1828). Furthermore, skin inflammation may be the representation of systemic diseases.

Skin inflammatory responses involve several cell types (T-cells and NK cells) and cytokines such as interferon gamma.

Among skin inflammatory diseases are psoriasis, allergic contact dermatitis, atopic dermatitis, cutaneous graft versus host disease, cutaneous cell lymphoma, among others.

### Psoriasis

They are associations between psoriasis and various loci of the immune system, such as the TH17 pathway (IL12B, IL23A, IL23R, TRAF3IP2, TYK2), innate immunity [NFκB and IFN] signaling pathways (TNFAIP3, TNIP1, NFKBIA, REL, TYK2, IFIH1, IL23RA) and β-defensin, the TH2 pathway (IL4, IL13), and adaptive immunity involving CD8 T cells (ERAP1, ZAP70). Thus cytokines (Th1, Th17 and Th22), chemokines, adhesion molecules, growth factors like NGF, neuropeptides, and specific T cell subpopulations along with their receptors all act in an integrated way to evolve into unique inflammatory and proliferative processes typical of psoriasis.

### Dermatitis

NK cells are involved in skin immune responses to haptens by secreting type 1 cytokines and inducing keratinocyte apoptosis. Most of the NK cells isolated from the skin of patients with allergic contact dermatitis showed a CD32CD162CD56high phenotype. This NK cell population also expressed NKG2A, intermediate to high levels of perforin, NKG2D, NKp44, and NKp46 but lacked NKp30 and killer immunoglobulin-related receptors. The CXCR31CCR61CCR51 chemokine receptor is an asset for homing into inflamed skin present in skin NK cells; however, they do not express CD62 ligand and CCR7 for lymph node homing.

### NK Cell-Mediated Contact Sensitivity

NK cell-mediated contact sensitivity (CS) in SCID and RAG1(−/−) mice but not in SCIDbeige mice, which have non-functional NK cells that lack NK cell granules. NK cell-mediated CS was transferred by liver mononuclear cells and the DX5(+) fraction of liver cells, confirming that NK cells mediate CS in the absence of T and B cells. Remarkably, NK cell-mediated CS was observed just 1 hr after immunization and was detectable as early as 30 min after challenge. Further, we examined cytokine requirements for NK cell-mediated CS, and found that liver mononuclear cells from interleukin-12(−/−), interferon-γ(−/−) and interferon-α receptor(−/−) donors fail to transfer NK cell-mediated CS to naive hosts.

### Palladium Allergy

Nickel, cobalt, and chromium are well known to be causal agents of allergic contact dermatitis. Palladium (Pd) can also cause allergic disease and exposure results from wide use of this metal in dental restorations and jewelry. Metal allergy is categorized as delayed-type hypersensitivity, and metal-responsive T cell clones have been isolated from allergic patients. Sequential adoptive transfer gradually increased the incidence and the intensity of Pd allergy, and CD8(+) T cells are responsible for the disease as CD8(+) T cell-depleted mice and β2-microglobulin-deficient mice did not develop Pd allergy. In addition, we found that draining lymph node cells skewed toward CD8(+) T cells in response to Pd challenge in 8th adoptive transferred recipient mice. The CD8(+) T cells expressed NKG2D, a costimulatory molecule involved in the production of IFN-γ. NKG2D ligand was also induced in Pd-injected tissues. Furthermore, both NKG2D ligand-transgenic mice, where NKG2D is downmodulated, and IFN-γdeficient mice showed impaired Pd allergy. Taken together, these results indicate that IFN-γ-producing NKG2D(+) CD8(+) T cells are responsible for Pd allergy and suggest that NKG2D is a potential therapeutic target for treatment of metal allergy.

NKG2D ligands (NKG2DL) are a family of proteins constituting two main groups.
The MHC class I-related chain A and B (MICA an MICB), which are encoded within the MHC.
The second group are the unique long 16 binding proteins (ULBP) of which, there are six distinct types (ULBP1-6).
Some viruses are able to avoid detection of ‘altered self’ by NK cells by employing mechanisms that cause down-regulation or retention of ULBP ligands within the cell.
NKG2DL can also be released from the cell surface in the soluble form by protease cleavage or released from the cell on the surface of exosomes and elevated levels have been widely reported associated with certain tumours or viral infections.
Interaction of NKG2D with soluble NKG2DL leads to blocking of the NKG2D receptor, or may cause this receptor to be downregulated, whichrenders the NK cell refractory to activation, a mechanism now recognised as having potential to allow tumour or virus progression by immune escape.

Secretion of NKG2DL may also be a natural mechanism of maternal-foetal tolerance.
Soluble NKG2D ligands have also been detected expressed on exosomes secreted by synciotrophoblast cells during human pregnancy.

NK cells are regulatory cells engaged in reciprocal interactions with dendritic cells, macrophages, T cells and endothelial cells. NK cells can thus limit or exacerbate immune responses. Although NK cells might appear to be redundant in several conditions of immune challenge in humans, NK cell manipulation seems to hold promise in efforts to improve hematopoietic and solid organ transplantation, promote antitumor immunotherapy and control inflammatory and autoimmune disorders.

Treatments currently available for NK cell mediated diseases such as dermatitis aim to control symptoms by reducing inflammation. Notably, steroids or topical immunosuppressant are used to control some of the symptoms due to cell-mediated immunity. However, corticosteroid based treatments have been shown to produce many side effects, and therefore, a lot of effort has been dedicated in developing additional therapeutic options for the treatments of dermatitis.

Also, there are clinically unrelated conditions, such as rheumatoid arthritis and Crohn's disease, sharing similar immune dysregulation. This has led to a shift in the management of IMIDs (immune-mediated inflammatory diseases) from one of organ-based symptom relief to mechanism-based treatment. The fact that anti-cytokine and anti-TNF antibody therapy (infliximab, etanercept, adalimumab, rituximab, abatacept, anakinra, alefacept and efalizumab) has been effective in treating multiple orphan inflammatory conditions confirms the IMID paradigm.

Cross-regulation between TNF and type I IFN has been postulated to play an important role in autoimmune diseases. Aberrant interferon gamma (IFN-γ) expression is associated with a number of autoinflammatory and autoimmune diseases. IFN-γ is produced predominantly by NK and NKT cells as part of the innate immune response, and by CD4 Th1 and CD8 cytotoxic T lymphocyte (CTL) effector T cells once antigen-specific immunity develops (Schoenborn& Wilson, 2007. Adv. Immunol. 96: 41-101).

IFN-γ plays an important role in regulating the immune system. It is a cytokine with pleiotropic effects and is thought to play a role in mediating various autoimmune diseases, as well as immune responses to infectious agents and cancer cells. IFN-γ regulates a variety of biological functions, such as antiviral responses, cell growth, immune response, and tumor suppression, and IFN-γ may mediate a variety of human diseases.

Thus, there is a need for agents or compositions that can modulate the biological activity of NK cells and IFN-γ.

### SUMMARY OF THE INVENTION

The examples of the present invention show that incubation of CBP (cord blood plasma) with PBMCs (peripheral blood mononuclear cells) prevents IL-15 induced proliferation of NK and T cells, and also the suppression of IFN-γ production. The present invention relates to the use of the umbilical cord blood plasma and/or serum, excluding the cellular components, in the manufacture of cosmetic as well as pharmacological compositions for the treatment of NK cell-mediated diseases and/or IFN-γ mediated diseases.

In one aspect, the present invention relates to the use of cord blood plasma or serum for modulating NK cell activity and/or IFN-γ activity.

In a preferred embodiment of this aspect of the invention, the cord blood plasma is human cord blood plasma.

In a second aspect, the invention relates to cord blood plasma or serum for use in medicine. In a preferred embodiment of this aspect, the invention relates to cord blood plasma or serum for use in the prevention, treatment or amelioration of NK cell-mediated diseases and/or IFN-γ mediated diseases. In another preferred embodiment of this aspect of the invention, the cord blood plasma is human cord blood plasma.

In a preferred embodiment of this second aspect of the invention, the NK cell-mediated disease and/or IFN-γmediated disease is an immune-mediated inflammatory disease, an autoimmune diseases, or an inflammatory disorder.

In another preferred embodiment, the NK cell-mediated and/or IFN-γmediated disease is selected from the group consisting of: lupus erythematosus, scleroderma, sclerodermoid disorders, vasculitis syndromes, occlusive vasculopathies, autoinflammatory syndromes, syndromes from innate immunity dysregulation (eghidradeitissuppurativa, pustular psoriasis), neutrophilicdermatoses, psoriasis, cardio-metabolic risk of chronic inflammation, atopic dermatitis, chronic itch, febrile dermatoses, psoriatic arthrithis, autoimmune bollous diseases, eosinophilicdermatoses, atopic eczema, urticaria, Bechet's disease, neutrophilicdermatoses, hidradenitissuppurativa, pustular psoriasis, autoimmune bullous diseases, chronic hepatitis B virus infection, sepsis induced by Streptococcus pneumoniae or Escherichia coli, diabetes induced by coxsackie virus B4, arthritis, sarcoidosis, collagenoses, rheumatism, hemolytic anemia, immune form of idiopathic thrombocytopenic purpura, eczemas, nephritis, myasthenia gravis, Hashimoto disease, autoimmune diseases of the organs of sight and hearing, multiple sclerosis, Meniere's disease, Parkinson's disease, pemphigus, schizophrenia, Crohn's disease or any combinations thereof.

In another preferred embodiment, the NK cell-mediated disease and/or IFN-γmediated disease is allograft rejection.

In another preferred embodiment, the NK cell-mediated disease and/or IFN-γmediated disease is graft versus host disease. In another more preferred embodiment, the graft versus host disease is the cutaneous graft versus host disease.

In another preferred embodiment, the NK cell-mediated disease and/or IFN-γmediated disease is a skin inflammatory disease. More preferably, the skin inflammatory disease is selected from the list consisting of: psoriasis, allergic contact dermatitis, atopic dermatitis, cutaneous graft versus host disease, cutaneous cell lymphoma, metal allergy, lichen planus, or any combinations thereof.

More preferably, the immune-mediated inflammatory disease, an autoimmune disease, or an inflammatory disorder is not keratoconjunctivitis.

Still more preferably, the skin inflammatory disease is psoriasis.

In a third aspect, the invention relates to a composition, hereinafter composition of the invention, comprising cord blood plasma or serum for use in medicine.

In a fourth aspect, the invention relates to a composition comprising cord blood plasma, or the composition of the invention, for use in the prevention, treatment or amelioration of NK cell-mediated disease and/or IFN-γmediated disease as described in the second aspect of the invention.

In a preferred embodiment, the composition of the invention further comprises a pharmaceutically acceptable carrier. In another preferred embodiment, the composition of the invention further comprises another active ingredient. More preferably, the composition of the invention is a pharmaceutical composition.

In a fifth aspect, the invention relates to a pharmaceutical form, in the following the pharmaceutical form of the invention, comprising the composition of the invention. In a preferred embodiment of this aspect of the invention, the pharmaceutical form of the invention is selected from the list comprising: poultice, ointment, paste, cream, solution, suspension, emulsion, lotion, liniment, gel, hydrogel, hydrocolloid, foam, spray, powder, or any combination thereof. In a more preferred embodiment, the pharmaceutical form of the invention is a poultice. In a much more preferred embodiment of the invention, the pharmaceutical form of the invention is selected from a solution, a suspension or an emulsion.

In a sixth aspect, the invention relates to the pharmaceutical form of the invention for use in the prevention, treatment or amelioration of NK cell-mediated disease and/or IFN-γmediated disease as described in the second aspect of the invention.

### BRIEF DESCRIPTION OF THE FIGURES

FIG. 1. Detection of NKD2D ligands (sMIC A/B and sULBP 1/2) in blood plasma from adult and in Umbilical cord plasma, the amounts of sMIC A/B and sULBP 1/2 are significantly higher in Umbilical cord plasma.

FIG. 2. Schematic of experimental conditions and procedure.

FIG. 3. CFSE proliferation assay. IL-15 activated PBMCs incubated with CB plasma dilutions were compared with cells incubated with media only at days 2, 5 and 7. Upper panel: CD56+ CD3− gated NK cells and lower panel CD3+CD56− gated T cells. Proliferation in the presence of CB plasma dilutions (Bluedark line) was compared to media only at each time-point.

FIG. 4. Histograms of percent maximum CFSE MFI at days 5 and 7 for CD56+CD3− NK cells. Error bars represent median±range. Statistical analysis was performed using Mann Whitney U test comparing CB dilutions with each other or basic media only (statistically significant ***P<0.0005, *P<0.02).

FIG. 5. Percentage of CD3− CD56+(NK), CD3+CD56+(NKT), CD3− CD56+(NKdim), CD3− CD56+(NKbri) and CD3+CD56− (T) cells staining negative for Annexin V and 7AAD after 24, 48 or 72 hours incubation with CB plasma dilutions or media only containing IL-2 at 200 I.U. per 200 ul culture. Error bars represent median and range and statistical analysis was performed using Mann Whitney U test (statistically significant ***P<0.0008, **P<0.006, *P<0.05).

FIG. 6. Percentage of NKdim and NKbri cells after 24, 48 or 72 hours incubation with CB plasma dilutions or media only containing IL-2 at 200 I.U. per 200 ul culture. Lower panels represent results with one CB plasma sample at various concentrations after 24 hours incubation. Error bars represent median and range and statistical analysis was performed using Mann Whitney U test (statistically significant ***P<0.0009, **P<0.009, *P<0.05).

FIG. 7. Percentage of NKT (CD56+CD3+) and T cells (CD56− CD3+) after 24, 48 or 72 hours incubation with CB plasma dilutions or media only containing IL-2 at 200 I.U. per 200 ul culture. Error bars represent median and range and statistical analysis was performed using Mann Whitney U test (statistically significant *P<0.05).

FIG. 8. Percentage of CD3− CD56+(NK), CD3− CD56+(NK bright), CD3− CD56+(NK dim), CD3+CD56+(NKT) and CD3+CD56− (T) cells staining negative for Annexin V and 7AAD after 24, 48 or 72 hours incubation with CB plasma dilutions or media only containing IL-2 at 200 I.U. per 200 ul culture. Error bars represent median±range.

FIG. 9. Percentage of specific K562 lysis after incubation of IL-15 activated NK cells (n=3) with either media only or differing concentrations of cord blood plasma for 24 or 72 hours and activated PBMCs (n=4) incubated for 48 hours with CB plasma or media. Narrow bars represent median values and statistical analysis was performed using Mann Whitney U test comparing CB dilutions with basic media only (statistically significant *P<0.02, **P<0.004, ***P=0.0001).

FIG. 10. Comparison of K562 specific lysis by resting and activated NK cells after 24 and 48 hours incubation with cord blood plasma dilutions or media only. The narrow bar represents median percent specific lysis (A and B) and statistical analysis was performed using Mann-Whitney U test (statistically significant ***P=0.0005, **P<0.01).

FIG. 11. Percentage of specific K562 lysis after 24 and 48 h incubation of resting isolated peripheral NK cells with either media (n=3) or differing concentrations of cord blood plasma (CBP, n=4). The experiment was repeated three times with different peripheral NK cell donors. Narrow bars represent median with interquartile range. *P≦0.05, **P≦0.01, ***P≦0.001, Mann Whitney u test.

FIG. 12. Percentage of specific K562 lysis, assessed by Cr51 release, after incubation of IL-15 activated, isolated peripheral NK cells with either media only (n=3) or differing concentrations of cord blood plasma (CBP, n=4) for 24, 48 or 72 h. The experiment was repeated three times with different NK cell donors. Line plots represent mean percent specific lysis (±SEM) at baseline (0 h) and after 24, 48 and 72 h (Kruskal-Wallis test). Scatter plots show median levels with interquartile range, comparing CBP dilutions with media only (Mann-Whitney u test). *P≦0.05, **P≦0.01, ***P≦0.001.

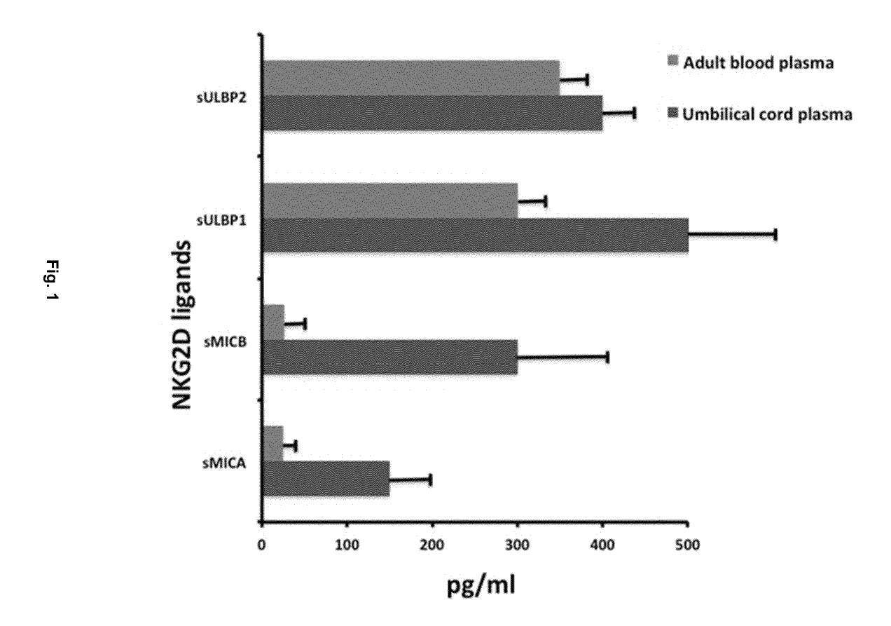

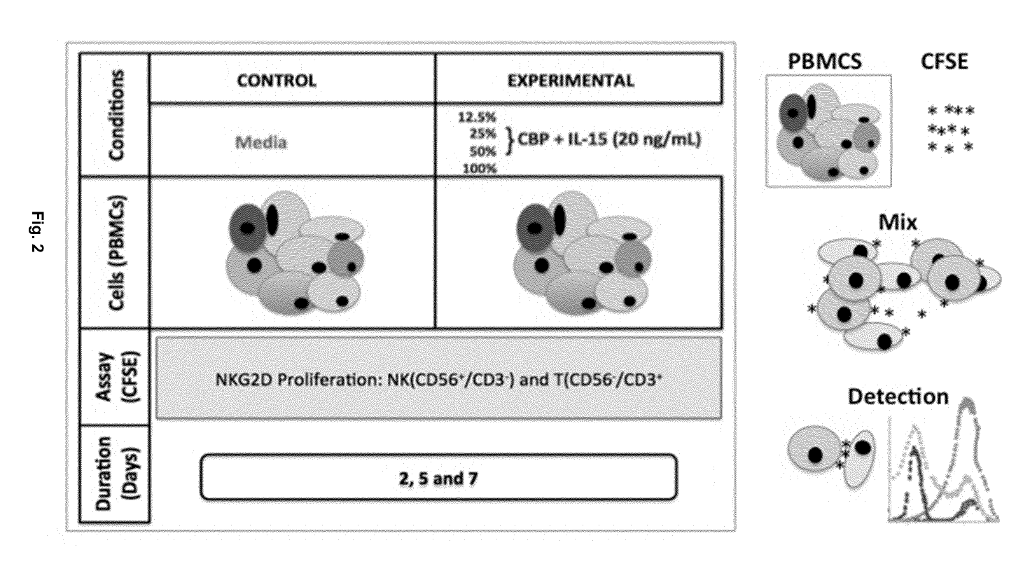

### DETAILED DESCRIPTION OF THE INVENTION

The present invention shows that CB plasma contains soluble NKG2D ligands and that the incubation of PBMCs with CB plasma results in decreased cytotoxicity, decreased proliferation and inhibition of IFNγ production by NKG2D bearing cells, in particular, NK cells. Interaction of NKG2D with soluble NKG2D ligand leads to blocking of the NKG2D receptor, and renders NKG2D bearing cells refractory to activation and inhibits cell functions. Notably, this is a mechanism naturally employed by tumor cells or viruses such as CMV to escape the immune system.

In one aspect, the present invention relates to the use of cord blood plasma or serum for modulating NK cell activity and/or IFN-γ activity.

In a preferred embodiment of this aspect of the invention, the cord blood plasma is human cord blood plasma.

### Medical Uses of the Invention

Taken together, the results of the invention suggest that CB plasma acts as a “natural immunosuppressant” and has similar properties to immunosuppressive drugs that are currently used to treat immune-mediated inflammatory disease, such as dermatitis.

Then, in a second aspect, the invention relates to cord blood plasma or serum for use in medicine. In a preferred embodiment of this aspect, the invention relates to cord blood plasma or serum for use in the treatment of NK cell-mediated diseases and/or IFN-γmediated diseases. In another preferred embodiment of this aspect of the invention, the cord blood plasma is human cord blood plasma.

Since the 1970s (Skurkovich et al. 1974, 1975, 1977, 1991), it was proposed that anti-cytokine therapy could be beneficial in treating a wide variety of autoimmune diseases and diseases of supposed autoimmune genesis, in which disturbed IFN synthesis is a common mechanism of pathology; these included, but is not limited, prolongation of skin allografts, collagenoses, rheumatism, hemolytic anemia, immune form of idiopathic thrombocytopenic purpura, eczemas, nephritis, myasthenia gravis, Hashimoto disease, autoimmune diseases of the organs of sight and hearing, multiple sclerosis, Meniere's disease, Parkinson's disease, pemphigus, schizophrenia and possibly some mental derangements, psoriasis and Crohn's disease. Skurkovich et al. (1974, 1975, 1977, 1991).

Then, in a preferred embodiment of this second aspect of the invention, the NK cell-mediated disease and/or IFN-γmediated disease is an immune-mediated inflammatory disease, an autoimmune disease, or an inflammatory disorder.

More preferably, the NK cell-mediated disease and/or IFN-γmediated disease is selected from the group consisting of: lupus erythematosus, scleroderma, sclerodermoid disorders, vasculitis syndromes, occlusive vasculopathies, autoinflammatory syndromes, syndromes from innate immunity dysregulation (eghidradeitissuppurativa, pustular psoriasis), neutrophilicdermatoses, psoriasis, cardio-metabolic risk of chronic inflammation, atopic dermatitis, chronic itch, febrile dermatoses, psoriatic arthrithis, autoimmune bollous diseases, eosinophilicdermatoses, atopic eczema, urticaria, bechet diseases, neutrophilicdermatoses, hidradenitissuppurativa, pustular psoriasis, autoimmune bullous diseases, chronic hepatitis B virus infection, sepsis induced by Streptococcus pneumoniae or Escherichia coli, diabetes induced by coxsackie virus B4, arthritis, sarcoidosis, collagenoses, rheumatism, hemolytic anemia, immune form of idiopathic thrombocytopenic purpura, eczemas, nephritis, myasthenia gravis, Hashimoto disease, autoimmune diseases of the organs of sight and hearing, multiple sclerosis, Meniere's disease, Parkinson's disease, pemphigus, schizophrenia, Crohn's disease or any combinations thereof.

NK cells often function as potent effector cells in rejection of allogeneic bone marrow cells (Kean et al., 2006. Am. J. Transplant. 6:292-304) and solid organ transplants (McNerney et al., 2006. Am. J. Transplant. 6:505-513; Uehara et al., 2005. J. Immunol. 175:3424-3430). Decreasing NK cell activity is therefore useful, for example, in the treatment of bone marrow cell allograft rejection.

In another embodiment, the NK cell-mediated disease and/or IFN-γmediated disease is allograft rejection, and more preferably, bone marrow allograft rejection, solid organ rejection, tissue rejection, for example cornea and allogenic cellular medicament rejection.

The morbidity and mortality associated with graft-host-disease (GVHD) is a significant obstacle to the greater use of allogeneic stem cell transplantation. Donor T cells that predominantly differentiate into Th1/Tc1 T cells, generate pro-inflammatory cytokines such as IFN-γ mediate GVHD.

Increased serum levels of IFN-γ are associated with acute graft-versus-host disease (GVHD), and lymphocytes from animals with GVHD secrete significantly greater amounts of IFN-γ than lymphocytes from non-GVHD controls (Szebeni et al., 1994. Transplantation 58:1385-1393 31-35; Wang et al., 1995. Transplantation 60:481-490; Troutt et al., 1992. Immunol. Cell Biol. 70:51-57; Allen et al., 1993. Eur. J. Immunol. 23:333-337; Ferrara et al., 1996. Stem Cells 14:473-48). Additional evidence of a role for IFN-γ in experimental acute GVHD includes: priming of macrophages by IFN-γ during acute GVHD to produce inflammatory cytokines (Nestel et al., 1992. J. Exp. Med 175:405-413); induction of pathology in skin tissues and the gastrointestinal tract by IFN-γ(37,38); suppression of T lymphocyte function characteristic of acute GVHD by IFN-γ (39,40); prevention of acute GVHD when CD81 cells are incapable of IFN-γ production (41); and inhibition of acute GVHD by direct or indirect blockade of IFN-γ(37,42-44).

Then, in another preferred embodiment, the NK cell-mediated disease and/or IFN-γ mediated disease is graft versus host disease. Still more preferably, the graft versus host disease is the cutaneous graft versus host disease.

Anti-IFN-γ or anti-TNF-α may generally be universal treatments for Th-1 autoimmune diseases, particularly skin diseases (Skurkovich&Skurkovich, 2006. Cytokines as Potential Therapeutic Targets for Inflammatory Skin DiseasesErnst Schering Research Foundation Workshop Volume 56, 2006, pp 1-27). CB plasma could be used in a similar manner, ie as topical application on reactions sites in order to decrease skin inflammation. In addition, it has been shown that NK cells are involved in the skin immune reaction in diseases such as dermatitis or contact sensitivity. As all NK cells express NKG2D, it is expected that CB plasma will inhibit the functions of these cells very potently. The authors of the present invention use CB plasma as a way to modulate or inhibit immune responses in the case of autoimmune diseases and inflammation that targets the skin via the action of soluble NKG2D ligands it contains.

In another preferred embodiment the NK cell-mediated disease and/or IFN-γ mediated disease is a skin inflammatory disease, and more preferably the skin inflammatory disease is selected from the list consisting of: psoriasis, allergic contact dermatitis, atopic dermatitis, cutaneous graft versus host disease, cutaneous cell lymphoma, metal allergy, lichen planus, or any combinations thereof.

Still more preferably, the skin inflammatory disease is the psoriasis.

In another preferred embodiment, the inflammatory disease is not the keratoconjunctivitis.

### Composition of the Invention

The plasma is prepared from the collected cord blood plasma and mixed with sterile normal saline, injected or added to solid organs, in cosmentic preparations, added to skin or to cellular medicaments.

In a fourth aspect, the invention relates to a composition comprising cord blood plasma, or the composition of the invention, for use the treatment of NK cell-mediated disease and/or IFN-γ mediated disease as described in the second aspect of the invention.

In a preferred embodiment, the composition of the invention further comprises a pharmaceutically acceptable carrier. In another preferred embodiment, the composition of the invention further comprises another active ingredient. A gluococorticoid and/or mycophenolatemofetil, azathioprine, leflunomide, methotrexate, or an anti-malarial can be administered concurrently with the composition of the invention.

More preferably, the composition of the invention is a pharmaceutical composition.

In a fifth aspect, the invention relates to a pharmaceutical form, in the following the pharmaceutical form of the invention, comprising the composition of the invention. In a preferred embodiment of this aspect of the invention, the pharmaceutical form of the invention is selected from the list comprising: poultice, ointment, paste, cream, solution, suspension, emulsion, lotion, liniment, gel, hydrogel, hydrocolloid, foam, spray, powder, or any combination thereof. In a more preferred embodiment, the pharmaceutical form of the invention is a poultice. In a much more preferred embodiment of the invention, the pharmaceutical form of the invention is selected from a solution, a suspension or an emulsion.

In a sixth aspect, the invention relates to the pharmaceutical form of the invention for use in the prevention, treatment or amelioration of NK cell-mediated disease and/or IFN-γmediated disease as described in the second aspect of the invention. More preferably relates to the pharmaceutical form of the invention for topical use in the treatment of a skin inflammatory disease. In a preferred embodiment, the skin inflammatory disease the skin inflammatory disease is selected from the list consisting of: psoriasis, allergic contact dermatitis, atopic dermatitis, cutaneous graft versus host disease, cutaneous cell lymphoma, metal allergy, lichen planus, or any combinations thereof. Still more preferably, the skin inflammatory disease is the psoriasis.

### Definitions

“Cord blood” or “CB” means umbilical cord blood.

In a preferred embodiment, the plasma or serum is derived from patient's own umbilical cord blood (autologous). In another embodiment of the invention, the formulation comprises donated umbilical cord blood plasma (allogeneic).

The cord blood plasma may be derived from patient's own umbilical cord blood or from the umbilical cord blood of a related or non-related donor.

The term “NK cell-associated disease or disorder,” as used herein, refers generally to NKcell-mediated diseases or disorders as well as diseases or disorders characterized by insufficient NK cell activity.

The term “NK cell-mediated disease or disorder,” as used herein, refers to any disease or disorder having a pathology that is mediated, at least in part, by NK cell cytolytic and immunoregulatory activity. An example of such a disease or disorder is acute rejection of bone marrow cell (BMC) allografts. Such diseases or disorders are particularly amenable to certain treatment methods for inhibition NK cell activity, as described further herein.

“NK cell activity” as used herein refers to NK cell cytolytic and immunoregulatory activity. There are numerous assays wellknown to the skilled artisan for detecting and/or monitoring such activity, including but not limited to the assays described in the examples provided herein.

An “IFN-γ-mediated disease,” as meant herein, is a disease in which evidence from an in vitro or a non-human model system or from human patients indicates IFN-γ is likely to play a role in driving the course of the disease. Diseases that are included among “IFN-γ-mediated diseases” include, for example, diseases in which patient samples display elevated levels of a type I or II IFN or a type I-related “IFN signature” pattern of gene expression. See, e.g., Baechler et al. (2003), Proc. Natl. Acad. Sci. 100(5): 2610-2615; Bennett et al. (2003), J. Exp. Med. 197(6): 711-723. IFN-γ-mediated diseases include, for example, but are not limited to, SLE, discoid lupus, lupus nephritis, alopecia greata, Grave's disease, Sjogren's syndrome, antiphospholipid syndrome, rheumatoid arthritis, juvenile idiopathic arthritis, psoriasis, psoriatic arthritis, dermatomyositis, polimyositis, bacterial septicemia, antigen/antibody complex diseases (Arthus-like syndromes), anaphylactic shock, multiple sclerosis (MS), type I diabetes, thyroiditis, graft versus host disease, transplant rejection, atherosclerosis, immune-mediated hepatic lesions, autoimmune hepatitis, inflammatory bowel diseases such as Crohn's disease and ulcerative colitis, giant cell arteritis, uveitis, macrophage activation syndrome (MAS), hemophagocyticlymphohistiocytosis (HLH), macrophage activation syndrome (MAS), sarcoidosis, and scleroderma.

The term “autoimmune disease”′ refers to a condition in a subject characterized by cellular, tissue and/or organ injury caused by an immunologic reaction of the subject to its own cells, tissues and/or organs Illustrative, non-limiting examples of autoimmune diseases which can be treated with the cell population of the invention include alopecia areata, ankylosing spondylitis, antiphosphohpid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricalpemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritisnodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sjogren's syndrome, Good pasture's syndrome, stiff-man syndrome, systemic lupus erythematosus, lupus erythematosus, takayasu arteritis, temporal arteπstis/giant cell arteritis, ulcerative colitis, uveitis, vascuhtides such as dermatitis herpetiformisvasculitis, vitiligo, Wegener's granulomatosis, etc

The term “immunoregulatory agent” refers to an agent that inhibits or reduces one or more biological activities of the immune system. An immunoregulatory agent is an agent that inhibits or reduces one or more biological activities (e.g., the proliferation, differentiation, priming, effector function, production of cytokines or expression of antigens) of one or more immune cells (e.g., T cells)

The term “inflammatory disease” refers to a condition in a subject characterized by inflammation, e.g. chronic inflammation Illustrative, non-limiting examples of inflammatory disorders include, but are not limited to, rheumatoid arthritis (RA), Inflammatory Bowel Disease (IBD), asthma, encephalitis, chronic obstructive pulmonary disease (COPD), inflammatory osteolysis, allergic disorders, septic shock, pulmonary fibrosis (e g, idiopathic pulmonary fibrosis), inflammatory vacuhtides (e g, polyarteritisnodosa, Wegner's granulomatosis, Takayasu's arteritis, temporal arteritis, and lymphomatoidgranulomatosus), post-traumatic vascular angioplasty (e.g., restenosis after angioplasty), undifferentiated spondyloarthropathy, undifferentiated arthropathy, arthritis, inflammatory osteolysis, chronic hepatitis, and chronic inflammation resulting from chronic viral or bacterial infections

As used herein, the terms ‘disorder”’ and “disease” are used interchangeably to refer to a condition in a subject.

In particular, the term “‘autoimmune disease’” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease ‘is used interchangeably with the term “inflammatory disorder”’ to refer to a condition in a subject characterized by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders.

Treatment of an NK cell mediated disease and/or IFN-γ mediated disease, including an autoimmune disease, encompasses alleviation of at least one symptom of the disorder, a reduction in the severity of the disease, or the delay or prevention of progression to a more serious disease that occurs with some frequency following the treated condition. Treatment need not mean that the disease is totally cured. A useful therapeutic agent needs only to reduce the severity of a disease, reduce the severity of a symptom or symptoms associated with the disease or its treatment, or provide improvement to a patient's quality of life, or delay the onset of a more serious disease that can occur with some frequency following the treated condition.

A “therapeutically effective dose,” as meant herein, is a dose that is effective to decrease one or more observable symptoms of a disease or to delay onset or mitigate the symptoms of a more serious condition that often follows after the condition that a patient is currently experiencing. A therapeutically effective dose may, but need not necessarily, completely eliminate all symptoms of the disease.

In the present application, the term “pharmaceutical form” makes reference to a mix of one or more active principles, with or without additives, whose physical characteristics are suitable for its dosage, preservation, administration and bioavailability.

A “poultice” or “plaster” is a pharmaceutical form consisting of a solid or semisolid form containing the active principle or principles, as well as additives, extended on a piece of fabric, plastic or adhesive tape acting as a support and protection, further having an occlusive effect and a macerating action allowing direct contact with the skin, and it softens due to the body temperature.

An “ointment” is a pharmaceutical form consisting of a preparation having a soft consistency containing the active principle or principles and additives incorporated to a suitable base providing the consistency and mass. When applied, it adheres to the skin and mucus. This base may be fat-soluble or water-soluble, it is generally anhydrous or with a maximum content of 20% water. It is also called hydrophilic ointment when the base is washable or removable with water.

A “paste” is a pharmaceutical form consisting of a semi-solid form containing the active principle or principles and additives, made from a high concentration of insoluble powder (20 to 50 percent) in oily or aqueous bases, weak absorbent or abrasive combined with soap.

A “cream” is a pharmaceutical form consisting of a liquid or semi-solid preparation containing the active principle or principles and additives required for obtaining an emulsion, generally oil in water, with a water content over 20 percent.

A “solution” is a pharmaceutical form consisting of a liquid, transparent and homogeneous liquid obtained by dissolving the active principle or principles and the additives in water, and which is employed for external or internal use. In the case of injectable, ophthalmic and otic solutions, they must be sterile. The term “solution” includes dilutions.

A “suspension” is a pharmaceutical form consisting of a disperse system formed by two phases, which contain the active principle or principles and the additives. One of the phases, which is continuous or external, is generally a liquid or a semisolid, and a dispersed or internal phase is made by insoluble solids (active principles) which are dispersible in the external phase. In case it is injectable, it must be sterile.

An “emulsion” is a pharmaceutical form consisting of a heterogeneous system generally formed by two immiscible liquids, where the dispersed phase is formed by small globules distributed in a carrier in which they are immiscible. The dispersed phase is known also as the internal phase and the dispersion medium is known as the external or continuous phase. There exist emulsions like water/oil, or oil/water, which may be semi-solid or liquid. The active principle or principles and additives may be in the external or in the internal phase.

A “lotion” is a pharmaceutical form, which can be in the form of a solution, a suspension or an emulsion, containing the active principle or principles and additives, where the dispersing agent is generally water.

A “liniment” is a pharmaceutical form consisting of a liquid presentation, a solution or an emulsion, containing the active principle or principles and additives, where the carrier is aqueous, alcoholic or oily.

A “jelly” is a pharmaceutical form consisting of a semi-solid colloid containing the active principle or principles and additives, having a water-soluble base generally made by gums such as tragacanth gum; other bases are: glycerin, pectin, alginate, glycerin boron compounds, synthetic derivatives, or natural substances such as carboxymethyllcelulose.

A “gel” is a pharmaceutical form consisting of a semi-solid preparation containing the active principle or principles and additives, solid in a liquid such as water, alcohol or oil, such that the particles form a net trapped inside the liquid phase.

A “hydrogel” is a system in a colloidal state having a solid appearance, such as albumin coagulated by heat, gelatin gelled by cold, etc. A property of hydrogels is that they swell and increase their volume when they absorb water and substances dissolved therein, this property being common to all tissues of organisms formed by colloidal matter.

A “colloid” is a material formed by a dispersed phase (internal) and a dispersing phase (filler). When the dispersing phase is water, it is called “hydrocolloid”. They can coagulate (go from solution to solid gel) if the dispersing phase is abundant, and flocculate (go from gel to solution) when the dispersion phase is scarce.

A “foam” is a pharmaceutical form consisting of a semi-solid preparation formed by two phases: a liquid phase carrying the active principle or principles and additives, and a gaseous phase comprising a propulsion gas for causing the product to exit in the form of a cloud.

The term “effective amount,” in the context of treatment of a NK cell-associated disease or disorder and/or IFN-γmediated disease by administration of CBP to a subject as described herein, refers to an amount of such molecule that is sufficient to modulate an NK cell and/or IFN-γmediated response in the subject so as to inhibit the occurrence or ameliorate one or more symptoms of the NK cell-associated disease or disorder and/or IFN-γmediated disease. An effective amount of an agent is administered according to the methods of the present invention in an “effective regime.” The term “effective regime” refers to a combination of amount of the agent being administered and dosage frequency adequate to accomplish treatment or prevention of the disease or disorder.

Along the description and claims, the word “comprises” and variants thereof do not intend to exclude other technical features, supplements, components or steps. For persons skilled in the art, other objects, advantages and features of the invention will be understood in part from the description and in part from the practice of the invention. The following examples and drawings are provided by way of illustration and they are not meant to limit the present invention.

### Examples

The following specific examples provided in this patent document serve to illustrate the nature of the present invention. These examples are included only for illustrative purposes and must not be interpreted as limiting to the invention claimed herein. Therefore, the examples described below illustrate the invention without limiting the field of application thereof.

### Materials and Methods

### Collection of Blood Samples

Peripheral blood mononuclear cells (PBMCs) were isolated from the blood of healthy volunteers upon written informed consent by density-gradient centrifugation using Lympholyte® Ficoll-Hypaque solution (Cedarlane, Ontario, Canada). Purified NK cells were obtained by negative selection using the NK cell isolation kit II (Miltenyi, Germany) according to the manufacturer's instructions. CB samples were obtained either from the Anthony Nolan Cord Blood Bank (Nottingham, UK) or from Dr Alicia Esparza Clinic (MEDIMAR, Alicante) and processed within 24 h of collection. Plasma from healthy volunteers and CB was isolated using centrifugation with a repeated centrifugation step to remove contaminating cells. Plasma was then heat inactivated (in 1.5 ml Eppendorf tubes) for 15 min at 58° C.

### Cell Culture

PBMCs were plated at 200,000 cells/well in media containing 10% fetal calf serum (FCS) and IL-2 (200 IU) or with CBP dilutions (diluted with media) containing the same concentration of IL-2 for 24, 48 or 72 h. Activation of isolated NK cells required 5 d culture with IL-15 (20 ng/ml) and cells were then plated at 50,000/well with media or plasma dilutions containing IL-15 for 24, 48 or 72 h for use in cytotoxicity analysis. Resting, isolated NK cells for cytotoxicity analysis were plated at 50,000 cells/well with media or plasma dilutions, without cytokines for 24 or 48 h.

### Flow Cytometry

Four-color flow cytometry analysis was performed using 200,000 PBMCs or 50,000 isolated NK cells, where appropriate. Briefly, cells were labelled in PBS containing BSA (0.5%) for 10 min at 4° C. Antibodies were as follows: anti-CD3 (SK7), anti-CD56 (B159), anti-CD107a (HA4A3 or anti-isotype IgG1 MOPC-21 control), anti-NKG2D (BAT221 or anti-isotype IgG2a BB23-8E6-8C8 control). Apoptosis and cell death was assessed using Annexin V and 7-AAD. Analysis was performed using a FACSCaliber instrument (BD Biosciences) and FlowJo Ver. 6.4.7 (Tree Star Inc., OR, USA).

### ELISA Analysis

IFN-γ in cell culture supernatants stimulated with PMA/ionomycin was measured using Human IFN-γ ELISA Ready-SET-Go! (eBioscience) and soluble TGF-β in CB and HA plasma was detected using Human/Mouse TGF-β1 (2nd Gen) ELISA Ready-SET-Go! (eBioscience) according to manufacturer's instructions.

### Cytotoxicity Analysis

51Cr-release assay was performed to assess NK cell cytolytic activity using K562 as target cells pulsed with 100 μCi Na2 51CrO4 for 45 min (PerkinElmer, Cambridge, UK). Freshly isolated NK cells or activated NK cells cultured for 5 days with IL-15 were incubated with CB plasma dilutions or media only for 24, 48 or 72 h. Cells were then washed and added to the target cells at effector-to-target ratio of 5:1 in triplicate. 51Cr-release was assessed in the supernatant of each culture after 4 h. The percentage of specific lysis was calculated as (experimental release−spontaneous re-lease)/(maximum release−spontaneous release)×100. For experiments with NKG2D blocking, 20 μg/ml NKG2D antibody (clone 1D11; eBioscience) or 20 μg/ml IgG1 isotype control (BD Biosciences) was added to cell cultures for 1 h prior to incubation with target cells.

### Proliferation Analysis

PBMCs were re-suspended in PBS at 106 cells/ml and labeled with 2 μM carboxyfluorescein diacetate succinimidyl ester (CFSE; Invitrogen, CA, USA) for 10 min in the dark at 37° C. Cells were then washed twice with media and cultured for 7 days in the presence of 20 ng/ml IL-15 with media only or CBP dilutions. CFSE incorporation by NK and T cell gated populations was measured on days 2, 5 and 7.

### Statistical Analysis

Unpaired datasets were compared using Mann Whitney U, t test and Kruskal-Wallis tests and paired comparisons were tested using Wilcoxon ranked pairs test, as appropriate. Tests were two-sided. GraphPad Prism 5 software (GraphPad, CA, USA) was used to perform the analysis.

### Results

Incubation of CBP with PBMCs prevents IL-15 induced proliferation of NK and T cells. Using CFSE, we measured proliferation of NKG2D-bearing NK (CD56+CD3−) and T (CD56− CD3+) cells incubated for up to 7 days with media or CBP dilutions containing 20 ng/ml IL-15. The potential of T (CD56− CD3+) and NK (CD56+CD3−) cells for proliferation after pre-incubation of PBMCs with media or CB plasma was investigated using the CFSE assay.

Overall, while high proliferation and was observed after 7 days with media only and 12.5% CB plasma, there was virtually no proliferation of both cell types with all other concentrations of CB plasma, which was highly significant compared with media only (P<0.0005) as shown in Table 1 and FIG. 2.

For cells incubated with media, CD56+CD3− NK cells began to proliferate by day 5 but was significantly inhibited by 100% and 50% CBP with partial inhibition by 25% CBP but not 12.5% CBP. By day 7, proliferation was still significantly inhibited by higher concentrations of CBP (P<0.0001, comparing 100% CBP with 12.5% CBP) and partially by 25% CBP but proliferation of cells incubated with 12.5% CBP was equivalent to media only cultures. CD56− CD3+ T cells took longer to proliferate, which was evident by day 7 and virtually completely inhibited by all CBP dilutions except 12.5%, which was not significantly different from media only cultures (Table 1).

TABLE 1
NK cells (CD56+/CD3−) NK proliferation rate inhibition

| | | 2 days | 5 days | 7 days |
|---|---|---|---|---|
| CBL−IL-15 | 12.5% | (−) | (+) | (+) |
| | 25% | (−) | (−/+) | (+) |
| | 50% | (−) | (−) | (−) |
| | 100% | (−) | (−) | (−) |

(−) completelyinhibited
(−/+) partiallyinhibited
(+) proliferation

TABLE 2
Percent of maximum CFSE MFI gated on CD3−
CD56+ NK cells and on CD3+ CD56− T cells.

| | | % Max CFSE MFI gated cells | | | |
|---|---|---|---|---|---|
| | | 2 days | 5 days | 7 days | |
| Media | | 100 | 20 | 10 | |
| CBP+IL-15 | 12.5% | 100 | 30 | 9 | NK (CD56+/ |
| | 25% | 100 | 90 | 70 | CD3−) |
| | 50% | 100 | 98 | 98 | |
| | 100% | 100 | 98 | 98 | |
| Media | | 100 | 94 | 9 | |
| CBP+IL-15 | 12.5% | 100 | 91 | 10 | T (CD3+/ |
| | 25% | 100 | 92 | 89 | CD56−) |
| | 50% | 100 | 100 | 99 | |
| | 100% | 100 | 94 | 99 | |

TABLE 3
Percentage of divided cells gated on CD3−
CD56+ NK cells and on CD3+ CD56− T cells.

| | | % Divided gated cells | | | |
|---|---|---|---|---|---|
| | | 2 days | 5 days | 7 days | |
| Media | | 10 | 45 | 80 | NK |
| CBP+IL-15 | 12.5% | 10 | 35 | 60 | |
| | 25% | 10 | 15 | 20 | |
| | 50% | 10 | 10 | 11 | |
| | 100% | 10 | 10 | 10 | |
| Media | | 1 | 11 | 14 | T |
| CBP+IL-15 | 12.5% | 1 | 4 | 5 | |
| | 25% | 1 | 2 | 5 | |
| | 50% | 1 | 1 | 1 | |
| | 100% | 1 | 1 | 1 | |

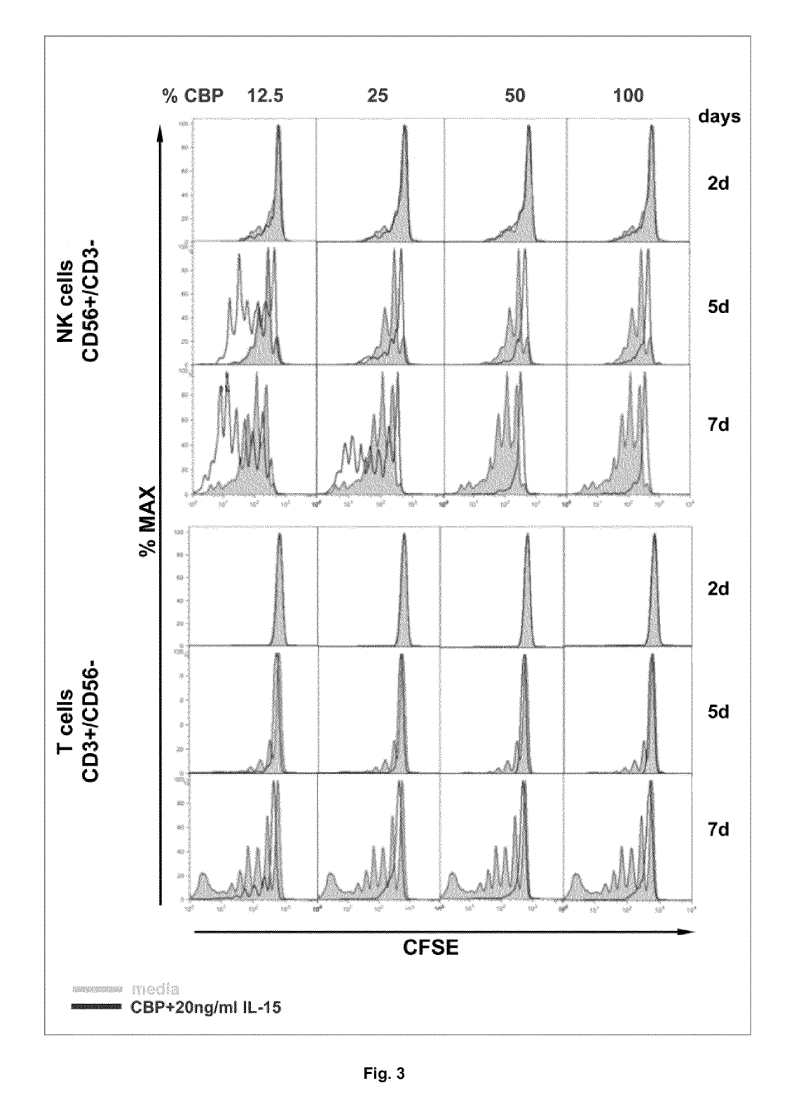

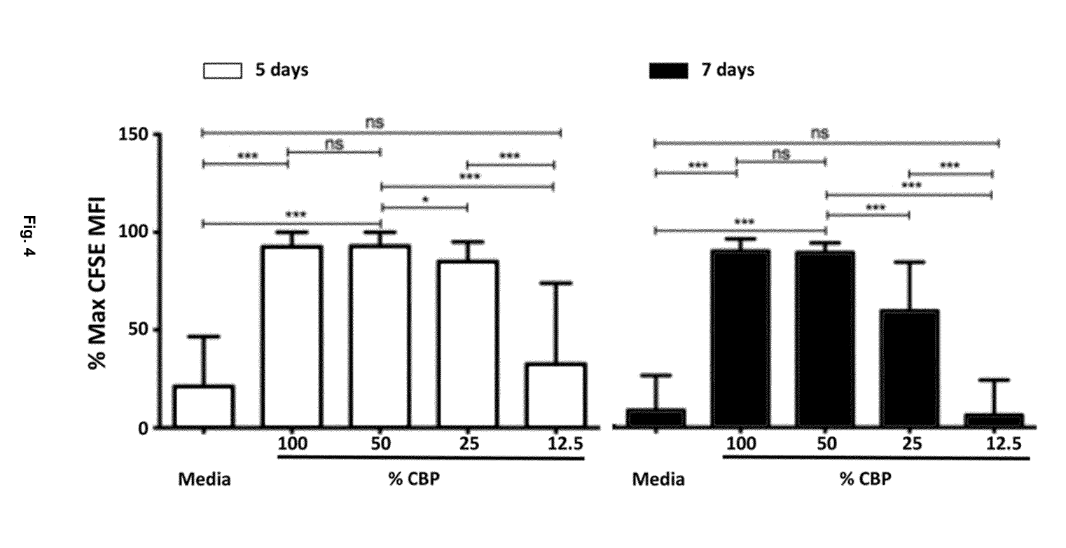

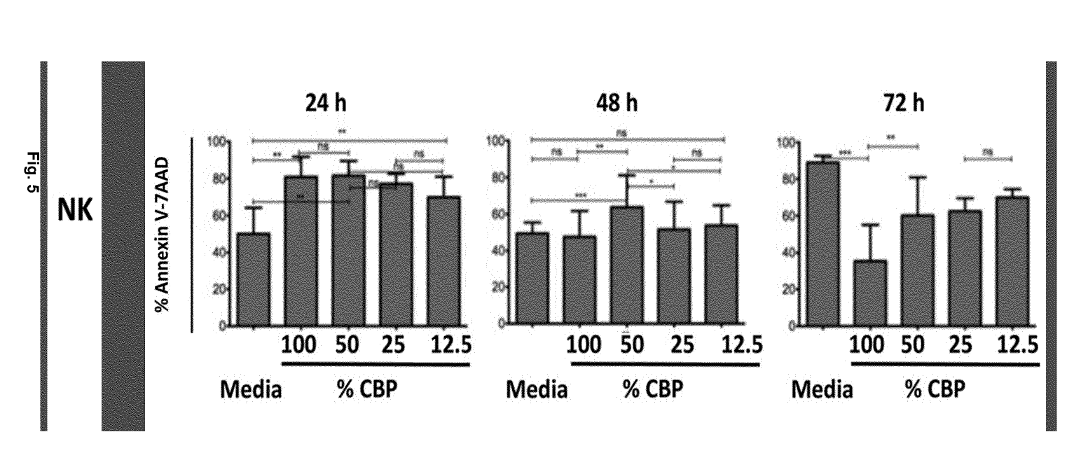

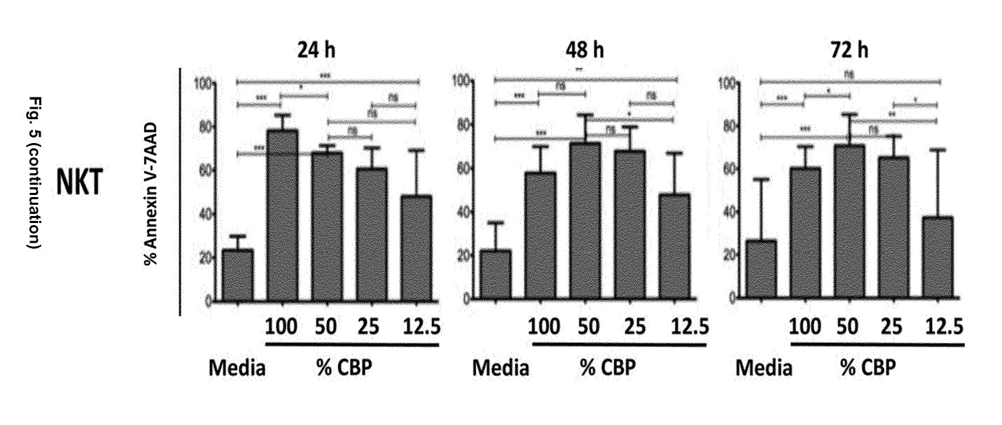

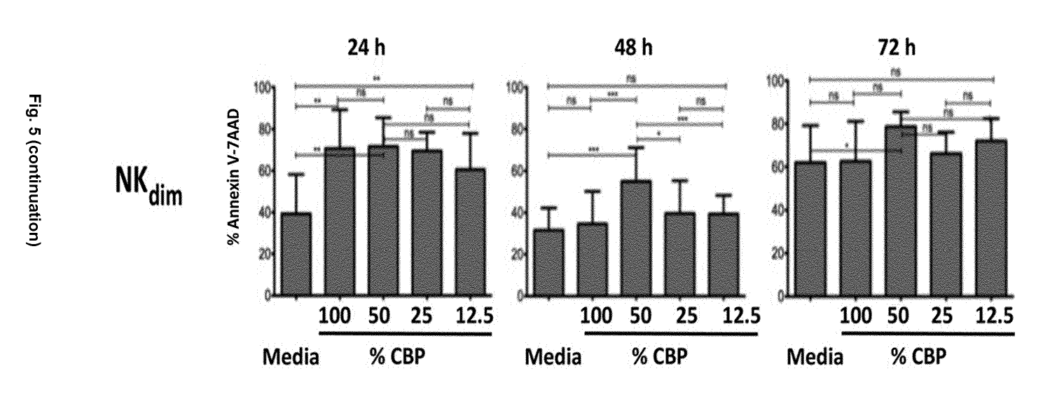

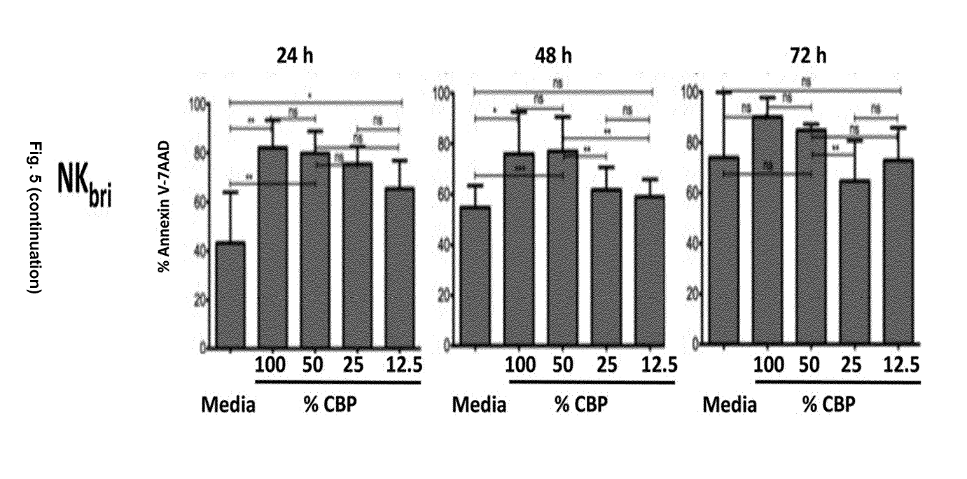

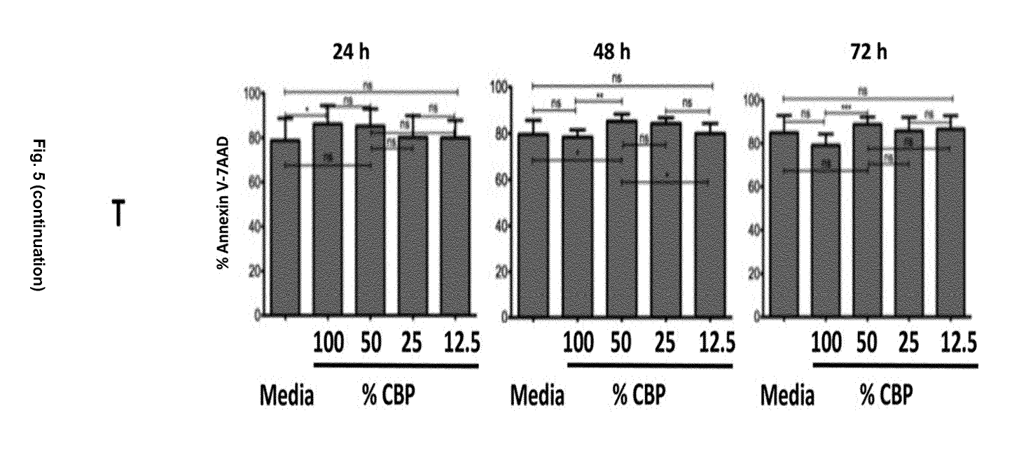

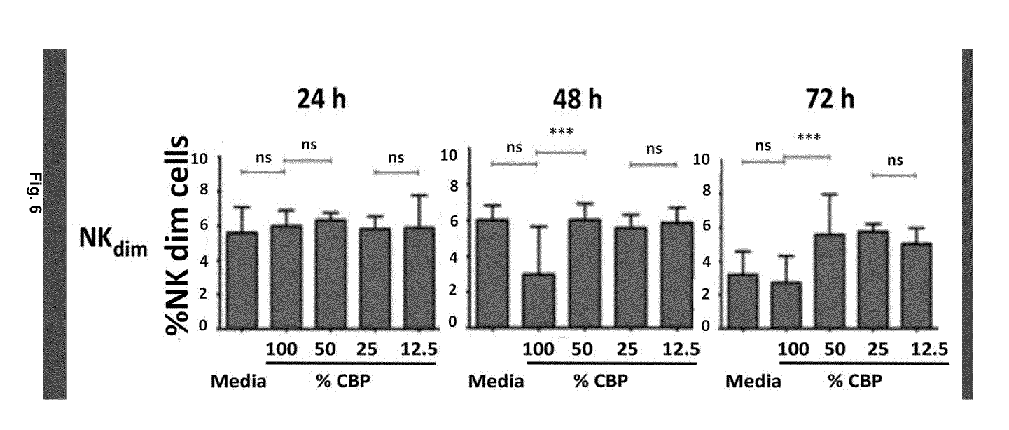

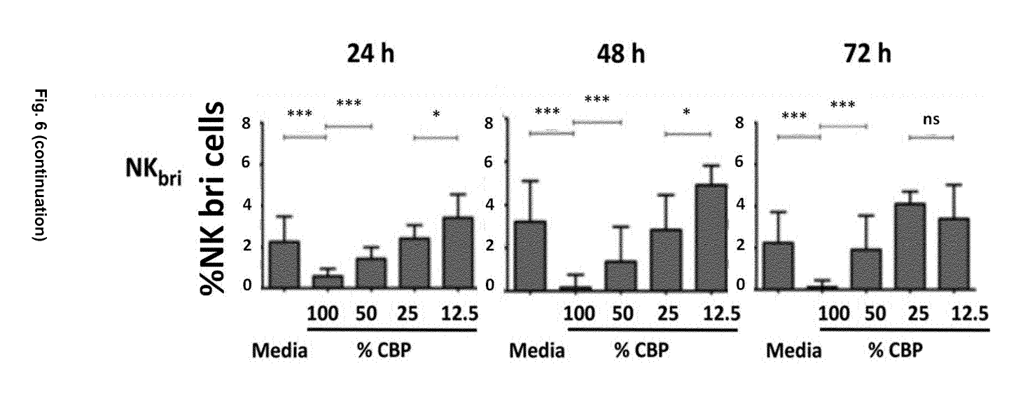

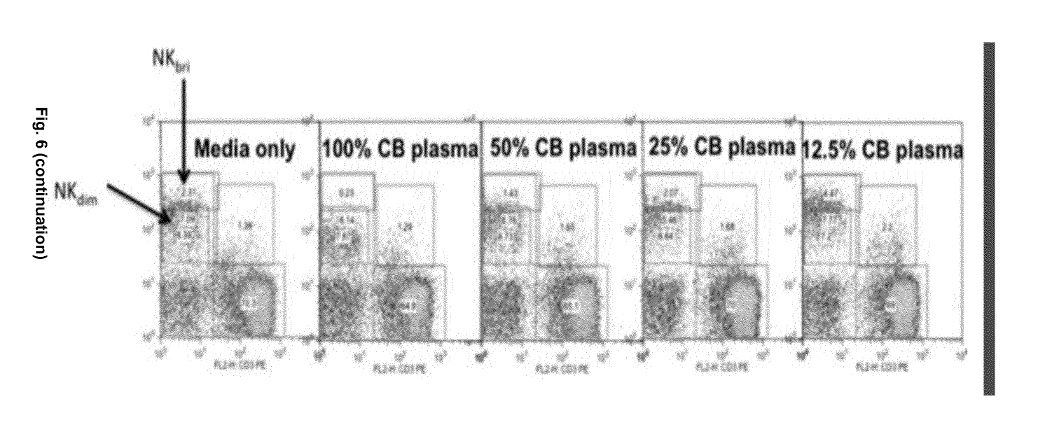

CBP increases viability of cultured healthy donor PBMCs and isolated resting and activated NK cells. It is possible that CB plasma may be toxic to both PBMCs and isolated resting and activated NK cells, therefore we repeated all culture conditions and, at the various time-points, measured staining of apoptosis marker Annexin V and cell death marker 7AAD. Results using PBMCs are reported in FIGS. 3-7 as percent Annexin V-7AAD-, which are live, healthy cells or as relative cell percentages. Overall, higher percentages were observed at all time-points when cells where incubated with CB plasma compared with media, indicating that the cultures are healthy. We are currently completing work with isolated, resting and IL-15 activated NK cells and preliminary data reveals similar results as for PBMCs. PBMCs cultured with IL-2 showed significantly higher viability of CD56dim and CD56bright cells with all CBP concentrations compared to media only. The same was also observed with CD56+CD3+ NKT cells only more striking, as media only cultures had relatively low median viability of 22.2±4.77−26.6±10.02% compared to around 50-70% where CBP dilutions were used. Although some significant differences were observed with CD56− CD3+ T cells, viability was around 80-90% in all cultures. The same pattern as PBMC-derived NK cells was observed with isolated resting CD56dim and CD56bright cells although this was not significant with CD56dim cells and viability was generally higher with isolated NK cells than PBMCs. Finally, a slightly different pattern emerged with isolated, IL-15 activated CD56dim and CD56bright cells. At lower CBP concentration, viability was comparable to media only but viability was increased, particularly for CD56dim cells, with higher concentrations of CBP. Overall, it is unlikely that the effects of CBP in terms of cellular function and cytotoxicity are attributable to cellular viability under the different experimental conditions.

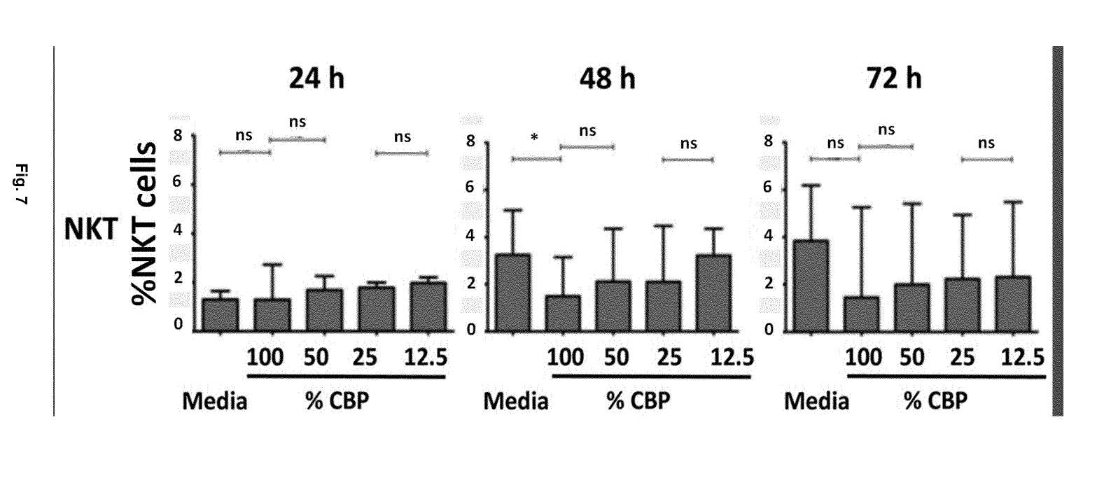

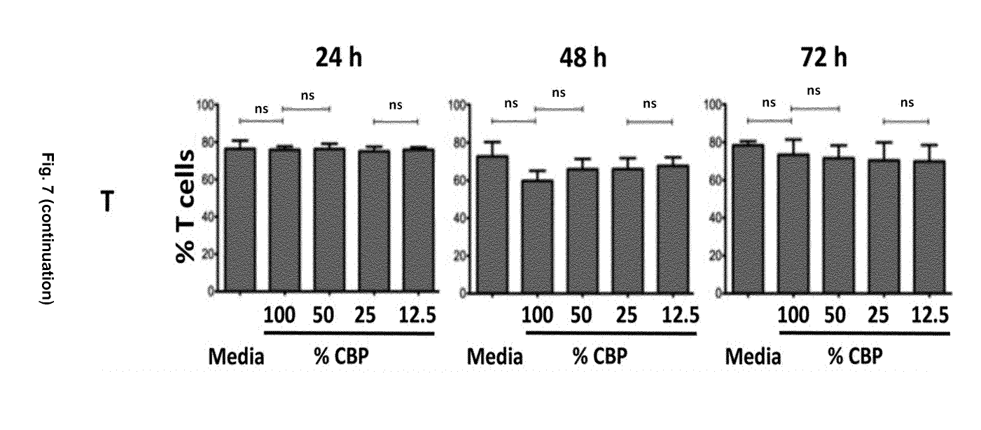

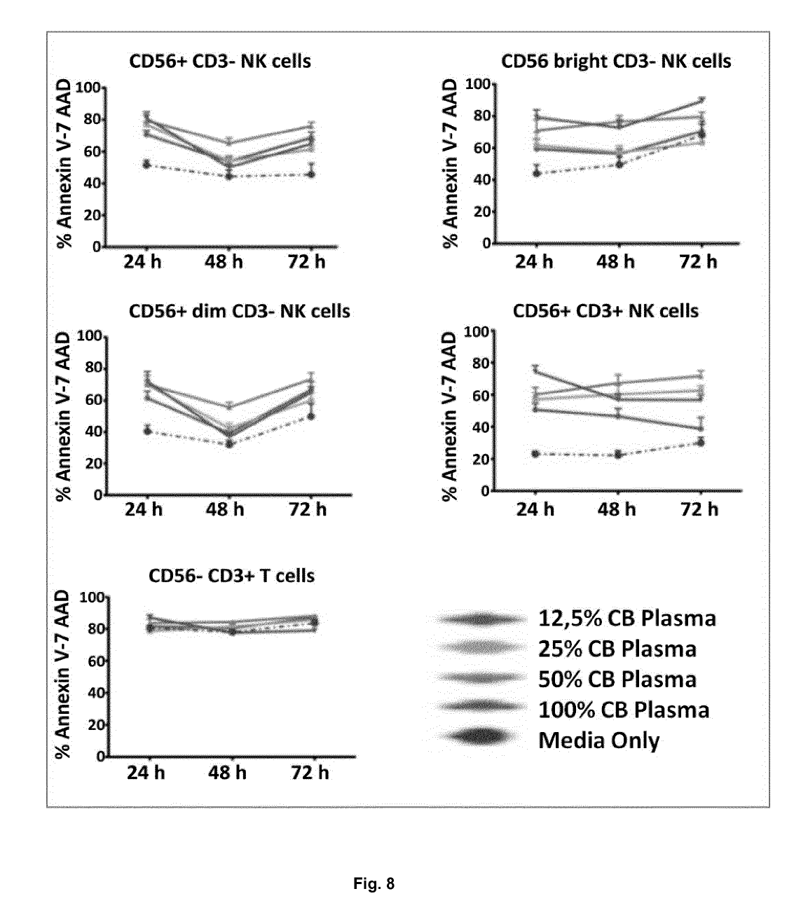

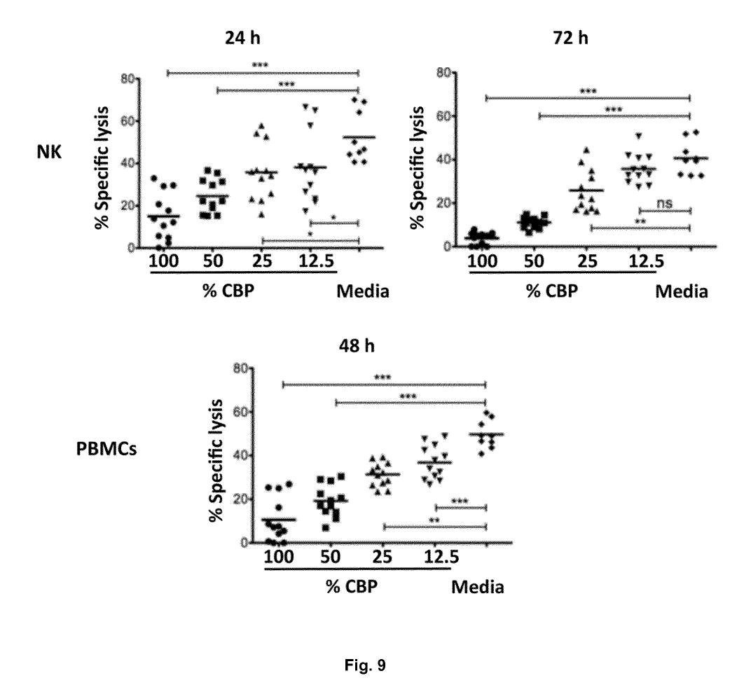

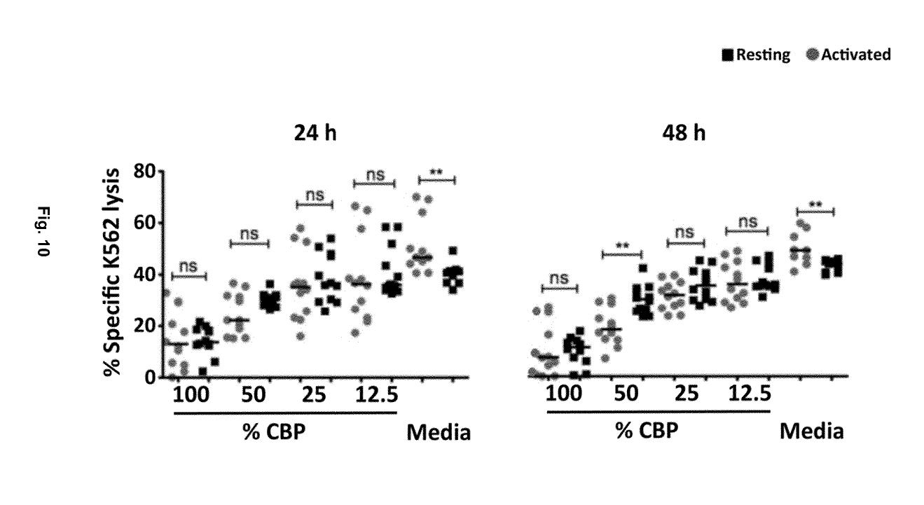

To ascertain whether the observed phenotypic characteristics were directly related to cellular function, we first determined levels of IFN-□ in supernatants of PBMC cultures derived from the previous experiments. Highly significant differences compared with media only were observed when cells were incubated with CB plasma in a dose-dependent manner, with virtually no IFN-γ production where 100% CB plasma was used (P<0.009, Mann Whitney U). The suppression of IFN-γ production by CB plasma continued over a 3-day period. Next, we investigated the capacity of resting (FIG. 9) and IL-15 activated (FIGS. 8 and 9), isolated NK cells to lyse K562 cells after incubation with media only or differing concentrations of CB plasma, in a chromium-release assay. This also resulted in a CB plasma dose-dependent reduction in percent specific K562 lysis, which was highly significant with higher concentrations of CB plasma with both resting and activated NK cells. Comparing percent specific lysis between resting and activated samples, significantly higher lysis was achieved with IL-15 activated media only cultures. Except for 50% CB plasma where significantly lower lysis was achieved with activated cells after 48 hours pre-incubation, there was no significant difference in lysis between resting and activated cells incubated with all concentrations of CB plasma after 24 and 48 hours as shown in FIG. 10.

Incubation of CBP with PBMCs prevents production of IFN-γ. To ascertain whether the observed phenotypic characteristics were directly related to cellular function, we first determined levels of IFN-γ in supernatants of PBMC cultures derived from the previous experiments. Compared with media only, highly significant dose-dependent differences were observed when cells were incubated with CBP, with virtually no IFN-γ production where 100% CBP was used, as shown in Fig. xxx. For example, after 48 h incubation, cells incubated with media produced 10.75±5.16 ng/ml (median±SD) compared with only 0.06±0.19 ng/ml where cells were incubated with pure CBP (P<0.0001) and 0.6±1.0 ng/ml with 50% CBP (P<0.0001). At the lowest CBP concentration of 12.5%, levels of IFN-γ were 3.28±3.16 ng/ml, which was less than a third of that produced with media only (P<0.001). The suppression of IFN-γ production by CBP continued over a 3-day period.

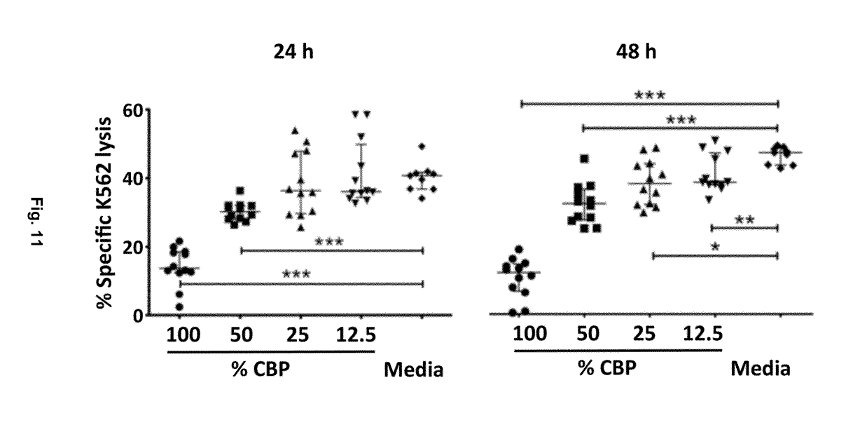

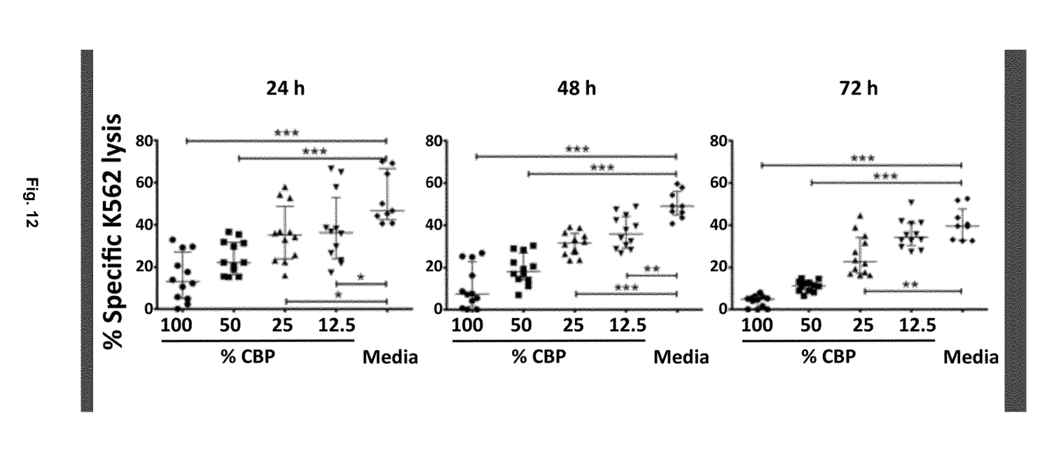

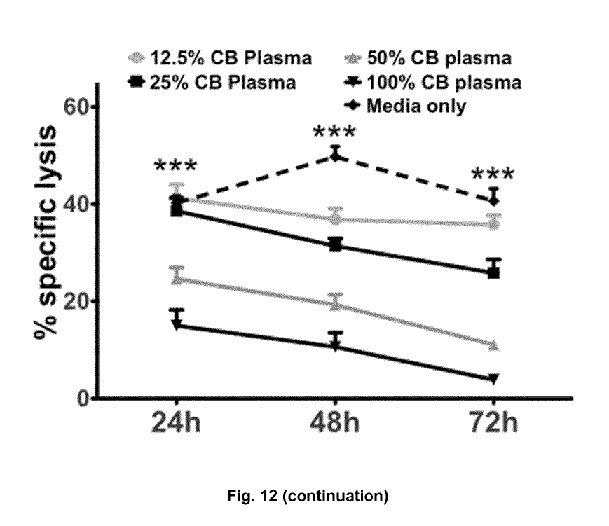

Incubation of resting and IL-15 activated NK cells with CBP significantly and dramatically reduces cytotoxicity. Next, we investigated the capacity of resting (FIG. 11) and IL-15 activated (FIG. 12) NK cells to lyse K562 cells after incubation with media only or differing concentrations of CBP, in a chromium-release assay. This also resulted in a CBP dose-dependent reduction in K562 lysis, which was highly significant with higher concentrations of CBP with both resting and activated NK cells. For example, after 48 h incubation of activated NK cells with media, specific lysis (%) was 49.06±6.38 (median±SD) compared with only 7.36±10.18 with 100% CBP (P=0.0001), 18.25±7.35 with 50% CBP (P=0.0001) and 35.93±7.76 with 12.5% CBP (P=0.0025).

Surprisingly, there was a general trend whereby K562 lysis suppression by CBP was enhanced when cells where activated rather than resting, although this was only significantly different with 50% CBP. After 24 h incubation with 50% CBP, the specific lysis for active and resting NK cells was 22.1±8.0 and 30.21±2.74, respectively (P=0.029; t test) and after 48 h, specific lysis was 18.25±7.35 and 30±5.52, respectively (P=0.0006; t test). As expected specific lysis was significantly higher with activated NK cells incubated with media only compared to resting cells. After 24 h incubation, specific lysis by activated cells was 46.68±10.95 compared with 40.74±4.3 for resting cells (P=0.0096; t test) and after 48 h, lysis was 49.06±6.38 and 43.77±2.33, respectively (P=0.0093; t test).

1. The use of cord blood plasma or serum for modulating NK cell activity and/or IFN-γ activity.

2. Cord blood plasma or serum or a preparation of exosomes or microvesicles from the same origin for use in the prevention, treatment or amelioration of NK cell-mediated disease and/or IFN-γmediated disease.

3. The cord blood plasma or serum for use in the prevention, treatment or amelioration of NK cell-mediated diseases and/or IFN-γmediated diseases according to claim 2, wherein the NK cell-mediated disease and/or IFN-γmediated disease is a immune-mediated inflammatory disease, an autoimmune diseases, or an inflammatory disorder.

4. The cord blood plasma or serum according to any one of claims 2-3, wherein the NK cell-mediated disease and/or IFN-γmediated disease is selected from the group consisting of: lupus erythematosus, scleroderma, sclerodermoid disorders, vasculitis syndromes, occlusive vasculopathies, autoinflammatory syndromes, syndromes from innate immunity dysregulation (eghidradeitissuppurativa, pustular psoriasis), neutrophilicdermatoses, psoriasis, cardio-metabolic risk of chronic inflammation, atopic dermatitis, chronic itch, febrile dermatoses, psoriatic arthrithis, autoimmune bollous diseases, eosinophilicdermatoses, atopic eczema, urticaria, bechet diseases, neutrophilicdermatoses, hidradenitissuppurativa, pustular psoriasis, autoimmune bullous diseases, chronic hepatitis B virus infection, sepsis induced by Streptococcus pneumoniae or Escherichia coli, diabetes induced by coxsackie virus B4, arthritis, sarcoidosis, collagenoses, rheumatism, hemolytic anemia, immune form of idiopathic thrombocytopenic purpura, eczemas, nephritis, myasthenia gravis, Hashimoto disease, autoimmune diseases of the organs of sight and hearing, multiple sclerosis, Meniere's disease, Parkinson's disease, pemphigus, schizophrenia, Crohn's disease or any combinations thereof.

5. The cord blood plasma or serum according to any one of claims 2-4, wherein the NK cell-mediated disease and/or IFN-γmediated disease is the allograft rejection.

9. The cord blood plasma or serum according to any one of claims 2-4, the NK cell-mediated disease and/or IFN-γ mediated disease is the graft versus host disease.

10. The cord blood plasma or serum according to claim 9, wherein graft versus host disease is the cutaneous graft versus host disease.

11. The cord blood plasma or serum according to any one of claims 2-4, wherein the NK cell-mediated disease and/or IFN-γ mediated disease is a skin inflammatory disease.

12. The cord blood plasma or serum according to claim 11, wherein the skin inflammatory disease is selected from the list consisting of: psoriasis, allergic contact dermatitis, atopic dermatitis, cutaneous graft versus host disease, cutaneous cell lymphoma, metal allergy, lichen planus, or any combinations thereof.

13. The cord blood plasma or serum according to any one of claims 10-11, wherein the skin inflammatory disease is the psoriasis.

14. A composition comprising cord blood plasma or serum for use in the prevention, treatment or amelioration of NK cell-mediated disease and/or IFN-γmediated disease according to any one of claims 2-13.

15. The composition according to claim 14, which is a pharmaceutical composition.

16. The composition according to any one of claims 14-15, further comprising a pharmaceutically acceptable carrier.

17. The composition according to any one of claims 14-16, further comprising another active ingredient

18. A pharmaceutical form comprising the composition according any one of claims 14-17.

19. The pharmaceutical form according to the preceding claim which is selected from the list comprising: patch, ointment, paste, cream, solution, suspension, emulsion, lotion, liniment, gel, hydrogel, hydrocolloid, foam, spray, powder, or any combination thereof.

20. The pharmaceutical form according to anyone of claims 18-19 for topical use in the treatment of a skin inflammatory disease as described in anyone of claims 11-13.
